(19) United States
(12) Patent Application Publication
(10) Pub. No.: US 2024/0353993 A1
(43) Pub. Date: Oct. 24, 2024

(54) EMOTION ESTIMATION METHOD, CONTENT DETERMINATION METHOD, RECORDING MEDIUM, EMOTION ESTIMATION SYSTEM, AND CONTENT DETERMINATION SYSTEM

(71) Applicant: Panasonic Intellectual Property Management Co., Ltd., Osaka (JP)

(72) Inventor: Shinichiro KURIHARA, Hyogo (JP)

(21) Appl. No.: 18/754,925

(22) Filed: Jun. 26, 2024

Related U.S. Application Data
(63) Continuation of application No. PCT/JP2022/036346, filed on Sep. 29, 2022.

(30) Foreign Application Priority Data
Jan. 18, 2022 (JP) 2022-006015

(51) Int. Cl. G06F 3/04847; G06F 3/16

(52) U.S. Cl. CPC G06F 3/04847; G06F 3/165

(57) ABSTRACT

An emotion estimation method includes: obtaining a first parameter indicating a user's subjective mood, a second parameter indicating the user's subjective excitement degree, and a third parameter indicating the user's subjective relaxation degree; estimating an emotion parameter indicating the user's emotion based on the obtained first parameter, second parameter, and third parameter; and outputting information related to the emotion parameter.

### CROSS REFERENCE TO RELATED APPLICATIONS

This is a continuation application of PCT International Application No. PCT/JP2022/036346 filed on Sep. 29, 2022, designating the United States of America, which is based on and claims priority of Japanese Patent Application No. 2022-006015 filed on Jan. 18, 2022. The entire disclosures of the above-identified applications, including the specifications, drawings and claims are incorporated herein by reference in their entirety.

### FIELD

The present disclosure relates to an emotion estimation method, a content determination method, a recording medium, an emotion estimation system, and a content determination system.

### BACKGROUND

Patent Document (PTL) 1 discloses a technique to obtain a user's biometric data and calculate a current emotional state value indicating the user's current emotional state based on the biometric data.

### CITATION LIST

### Patent Literature

PTL 1: Japanese Unexamined Patent Publication No. 2018-195043

### SUMMARY

### Technical Problem

The present disclosure provides an emotion estimation method and the like that can estimate a user's emotion based on the user's subjective mood.

### Solution to Problem

An emotion estimation method according to one aspect of the present disclosure includes: obtaining a first parameter indicating a subjective mood of a user, a second parameter indicating a subjective excitement degree of the user, and a third parameter indicating a subjective relaxation degree of the user; estimating an emotion parameter indicating an emotion of the user based on the first parameter obtained, the second parameter obtained, and the third parameter obtained; and outputting information related to the emotion parameter.

### Advantageous Effects

The emotion estimation method according to one aspect of the present disclosure can estimate the user's emotion based on the user's subjective mood.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1 is a block diagram illustrating an overall configuration including an emotion estimation system and a content determination system according to an embodiment.

FIG. 2 is a diagram illustrating an example of a first parameter input screen in an information terminal according to the embodiment.

FIG. 3 is a diagram illustrating an example of a second parameter input screen in the information terminal according to the embodiment.

FIG. 4 is a diagram illustrating an example of a third parameter input screen in the information terminal according to the embodiment.

FIG. 5 is a diagram illustrating an example of an affect grid according to the embodiment.

FIG. 6 is an explanatory diagram of the user's emotion by the emotion estimation system according to the embodiment.

FIG. 7 is an explanatory diagram of a prediction model used in the content determination system according to the embodiment.

FIG. 8 is a schematic diagram illustrating an example of the operations of the emotion estimation system and the content determination system according to the embodiment.

FIG. 9 is a flowchart illustrating an example of the operation of the emotion estimation system according to the embodiment.

FIG. 10 is a flowchart illustrating an example of the operation of the content determination system according to the embodiment.

FIG. 11 is an explanatory diagram of an example of playlist generation in the content determination system according to the embodiment.

FIG. 12 is an explanatory diagram of an example of playlist regeneration in the content determination system according to the embodiment.

### DESCRIPTION OF EMBODIMENTS

Hereinafter, exemplary embodiments will be described below with reference to the drawings. Note that each of the exemplary embodiments described below shows a general or specific example. Numerical values, shapes, materials, components, arrangement and connection modes of the components, steps, the order of the steps, and the like, which will be shown in the following exemplary embodiments, are only examples and are not intended to limit the present disclosure. Among the components in the following exemplary embodiments, components not recited in independent claims are described as optional components.

Note that each of the drawings is a schematic diagram and is not necessarily illustrated exactly. In the drawings, the same reference numerals are assigned to substantially the same components, and a duplicate description may be omitted or simplified.

### Embodiment

### 1. Configuration

First, configurations of an emotion estimation system and a content determination system according to an embodiment will be described. An emotion estimation system is a system for estimating the user's emotion based on the user's subjective mood, subjective excitement degree, and subjective relaxation degree. The content determination system is a system for determining content to be provided to the user based on the user's emotion estimated by the emotion estimation system.

In the embodiment, the content is, for example, music played back in a space where the user is present, or sound content including natural environment sound. The natural environment sound is, for example, a song of a bird, a cry of an insect, a sound of wind, or a sound of running water. The content needs only to be content that can induce the user from the current emotional state to a predetermined emotion by providing the content to the user, and is not limited to sound content. For example, the content may be image content including a still image or a moving image played back in a space where the user is present, or may be lighting content including the brightness or color temperature of lighting that illuminates the space where the user is present.

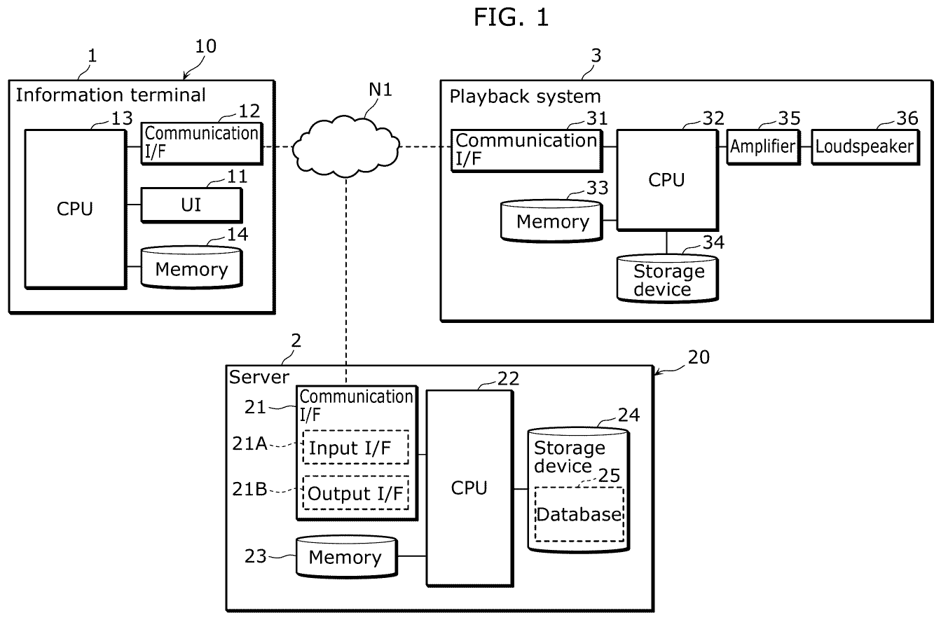

FIG. 1 is a block diagram illustrating an overall configuration including an emotion estimation system and a content determination system according to an embodiment. In the embodiment, emotion estimation system 10 is implemented by information terminal 1 used by a user. In the embodiment, content determination system 20 is implemented by server 2. In the embodiment, sound content determined by content determination system 20 is played back by playback system 3. Server 2 can communicate with each of information terminal 1 and playback system 3 via network N1 such as the Internet.

In FIG. 1, only one information terminal 1 is illustrated. In the embodiment, there are the same number of information terminals 1 as the number of a plurality of users. When there is only one user or when one information terminal 1 is shared by a plurality of users, there is only one information terminal 1.

In the embodiment, emotion estimation system 10, content determination system 20, and playback system 3 all target a user present in an office. In other words, emotion estimation system 10 estimates the emotion of the user present in the office. Content determination system 20 also provides sound content to the user present in the office based on the emotion of the users present in the office estimated by emotion estimation system 10. Playback system 3 plays back the sound content determined by content determination system 20, inside the office. Playback system 3 is installed, for example, in a predetermined place in the office. The predetermined place is a place where the user in the office can listen to the sound content played back by playback system 3. By way of example, the predetermined place may be on the ceiling in the center of the office or on a desk in the center of the office. Needless to say, emotion estimation system 10, content determination system 20, and playback system 3 may all target a user present in a space other than the office.

### [1-1. Configuration of Information Terminal]

First, the configuration of information terminal 1 will be described in detail. Information terminal 1 is a portable terminal such as a smartphone, a tablet terminal, or a lap-top personal computer. Note that information terminal 1 may be an installed terminal such as a desktop personal computer. In the embodiment, information terminal 1 is a smartphone.

When a predetermined application is installed, information terminal 1 functions as emotion estimation system 10. Information terminal 1 includes user interface 11, communication interface (I/F) 12, central processing unit (CPU) 13, and memory 14.

User interface 11 is a device that receives the user's operation and presents an image to the user. User interface 11 is implemented by, for example, an operation reception device, such as a touch panel, and a display device, such as a display panel. User interface 11 is an example of an input interface in emotion estimation system 10. Note that the means for receiving the user's operation in user interface 11 may be implemented by an audio input reception device such as a microphone. The means for presenting information to the user in user interface 11 may be implemented by an audio output device such as a loudspeaker.

The user inputs a first parameter, a second parameter, and a third parameter while viewing an input screen displayed on user interface 11. Thus, user interface 11 obtains the first parameter, the second parameter, and the third parameter. The input of the first parameter, the second parameter, and the third parameter will be described in detail in [Input of First Parameter, Second Parameter, and Third Parameter] to be described later.

Here, the first parameter is a parameter indicating the user's subjective mood. The mood may include, for example, depression, gloom, satisfaction, or joy. The second parameter is a parameter indicating the user's subjective excitement degree. The excitement degree may include whether or not the user is excited or fatigued. The third parameter is a parameter indicating the user's subjective relaxation degree. The relaxation degree may include whether the user is relaxed or tense. Each of the first parameter, the second parameter, and the third parameter is a parameter indicating the user's self-reported emotion.

Communication interface 12 is, for example, a wireless communication interface and communicates with server 2 via network N1 based on a wireless communication standard such as Wi-Fi (registered trademark). Communication interface 12 communicates with server 2 via network N1 to transmit a signal to server 2. This signal includes an emotion parameter indicating the user's emotion estimated by CPU 13, which will be described later. Communication interface 12 is an example of an output interface in emotion estimation system 10.

CPU 13 performs information processing related to the display of an image on user interface 11 and the transmission of a signal using communication interface 12. Further, CPU 13 performs information processing for estimating an emotion parameter indicating the user's emotion based on the first parameter, second parameter, and third parameter obtained by user interface 11. The information processing for estimating the emotion parameter will be described in detail in [Estimation of User's Emotion] to be described later.

The image display processing, signal transmission processing, and emotion parameter estimation processing described above are all implemented by CPU 13 executing a computer program stored in memory 14. CPU 13 is an example of the signal processing circuit of emotion estimation system 10.

Memory 14 is a storage device for storing various information necessary for CPU 13 to perform information processing, a computer program executed by CPU 13, and the like. Memory 14 is implemented by, for example, a semiconductor memory.

### [1-2. Configuration of Server]

Next, the configuration of server 2 will be described in detail. Server 2 includes communication interface 21, CPU 22, memory 23, and storage device 24.

Communication interface 21 is, for example, a wireless communication interface, and communicates with information terminal 1 via network N1 based on a wireless communication standard such as Wi-Fi (registered trademark) to receive a signal transmitted from information terminal 1. Communication interface 21 communicates with playback system 3 via network N1 based on a wireless communication standard such as Wi-Fi (registered trademark) to transmit a signal to playback system 3.

Communication interface 21 has functions of both input interface 21A and output interface 21B. Input interface 21A receives a signal transmitted from information terminal 1 to obtain the emotion parameter estimated by emotion estimation system 10. Input interface 21A is an example of an input interface in content determination system 20.

In the embodiment, input interface 21A further obtains a target parameter indicating a target emotion for the user. The target parameter is set in advance, for example, by the administrator of content determination system 20. The setting of the target parameter is executed, for example, by the information terminal used by the administrator. Input interface 21A receives the signal transmitted from the information terminal of the administrator to obtain the target parameter. Note that the target parameter may be set in advance by the user, for example.

Output interface 21B transmits a signal to playback system 3 to output information related to the sound content determined by CPU 22. In the embodiment, the information related to the sound content is a playlist that defines the order in which the sound content is played back by playback system 3. Note that the playlist may specify the order in which a plurality of sound contents are played back, or may specify that only one sound content is repeatedly played back. Output interface 21B is an example of an output interface in content determination system 20.

CPU 22 performs information processing related to the transmission and reception of signals using communication interface 21, and performs information processing for determining sound content based on the emotion parameter obtained by communication interface 21. In the embodiment, CPU 22 further refers to the target parameter in the information processing for determining the sound content. The information processing for determining the sound content will be described in detail in “Determination of Sound Content” to be described later.

CPU 22 executes a computer program stored in memory 23 to implement all of the signal transmission processing, signal reception processing, and sound content determination process described above. CPU 22 is an example of the signal processing circuit of content determination system 20.

Memory 23 is a storage device for storing various information necessary for CPU 22 to perform information processing, a computer program executed by CPU 22, and the like. Memory 23 is implemented by, for example, a semiconductor memory.

Storage device 24 is a device for storing database 25, which CPU 22 references when executing information processing to determine sound content. Storage device 24 is implemented by a semiconductor memory such as a hard disk or a solid-state drive (SSD). Database 25 will be described in detail in “Determination of Sound Content” to be described later.

### [1-3. Configuration of Playback System]

Next, the structure of playback system 3 will be described in detail. Playback system 3 includes communication interface 31, CPU 32, memory 33, storage device 34, amplifier 35, and loudspeaker 36. Communication interface 31 is, for example, a wireless communication interface and communicates with server 2 via network N1 based on a wireless communication standard such as Wi-Fi (registered trademark). Communication interface 31 communicates with server 2 via network N1 to receive a signal from server 2. This signal includes the playlist determined by content determination system 20.

CPU 32 performs information processing related to the reception of a signal using communication interface 31, and performs information processing for causing loudspeaker 36 to play back sound content according to the playlist obtained by communication interface 31. That is, CPU 32 sequentially reads the sound content specified by the obtained playlist from storage device 34, and causes loudspeaker 36 to play back a sound signal including the read sound content via amplifier 35. As a result, loudspeaker 36 plays back the sound content according to the order defined by the playlist. CPU 32 executes a computer program stored in memory 33 to implement both the signal reception processing and sound content generation processing described above.

Memory 33 is a storage device for storing various information necessary for CPU 32 to perform information processing, a computer program executed by CPU 32, and the like. Memory 33 is implemented by, for example, a semiconductor memory.

Storage device 34 is a device for storing a plurality of sound contents played back by loudspeaker 36. Storage device 34 is implemented by a semiconductor memory such as a hard disk or a solid-state drive (SSD).

Amplifier 35 amplifies the input sound signal and outputs the amplified sound signal to loudspeaker 36. In the embodiment, amplifier 35 has an up-sampling function that converts the sampling rate of the sound signal to a higher sampling rate. Note that amplifier 35 need not have the up-sampling function.

Loudspeaker 36 converts the sound signal amplified by amplifier 35 into sound and outputs the sound, thereby playing back the sound content based on the sound signal.

### [1-4. Input of First Parameter, Second Parameter, and Third Parameter]

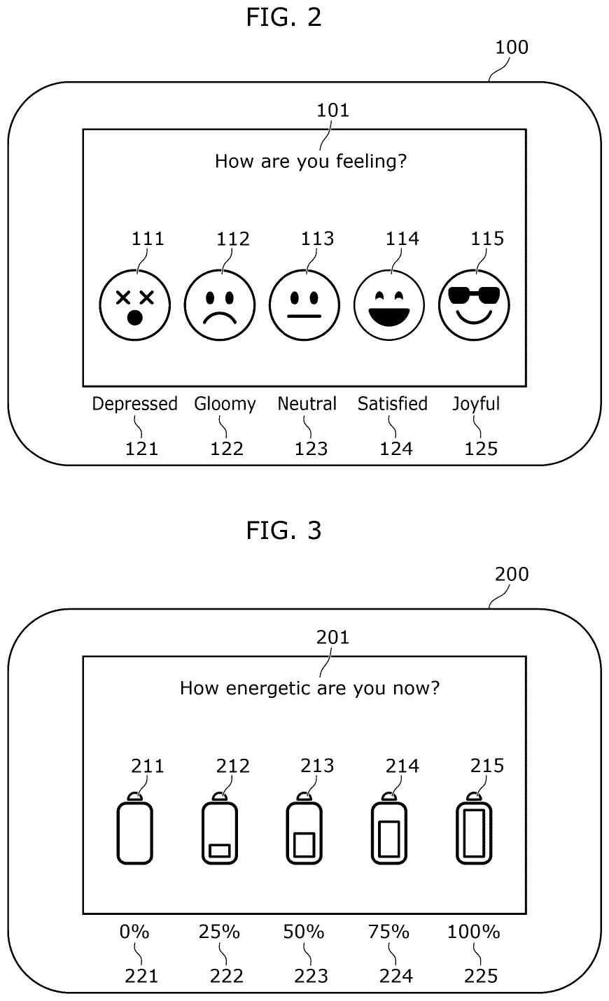

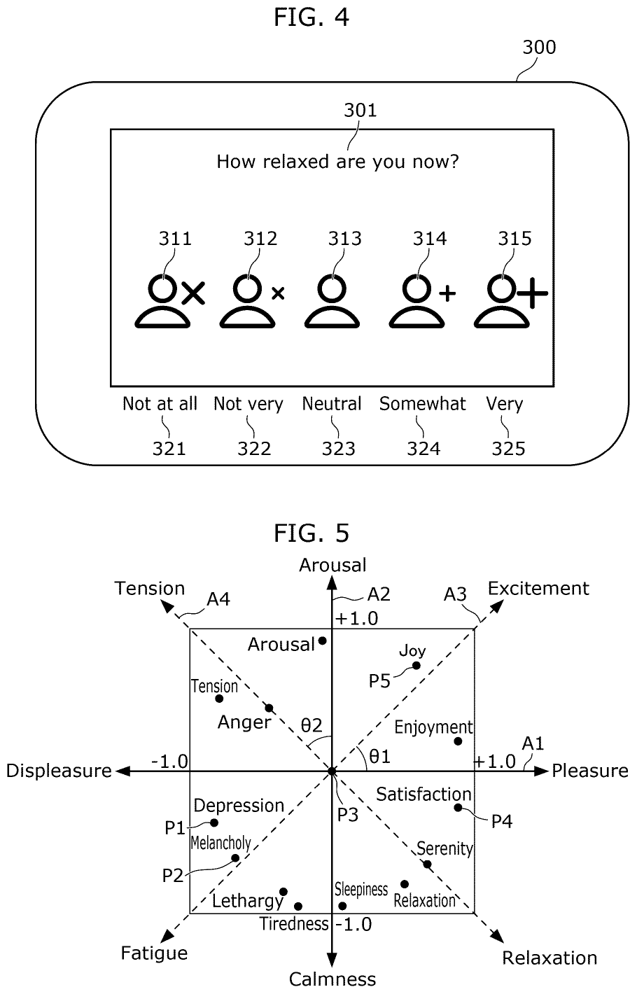

The input of the first parameter indicating the user's subjective mood, the second parameter indicating the user's subjective excitement degree, and the third parameter indicating the user's subjective relaxation degree will be described below with reference to FIGS. 2 to 4. FIG. 2 is a diagram illustrating an example of first parameter input screen 100 in information terminal 1 according to the embodiment. FIG. 3 is a diagram illustrating an example of second parameter input screen 200 in information terminal 1 according to the embodiment. FIG. 4 is a diagram illustrating an example of third parameter input screen 300 in information terminal 1 according to the embodiment.

In the embodiment, the first parameter, the second parameter, and the third parameter are all input by the user in user interface 11 of information terminal 1. Specifically, the user executes, for example, a predetermined application installed in information terminal 1. Then, first parameter input screen 100 illustrated in FIG. 2 is first displayed on user interface 11 of information terminal 1. This input screen 100 displays character string 101 representing a question to the user, “How are you feeling?”, a plurality of (five in this case) icons 111 to 115 representing the first parameter, and a plurality of character strings 121 to 125 describing the plurality of icons 111 to 115, respectively. In the lower portion of input screen 100, the icons and character strings are displayed in order from the left, with icon 111 and character string 121 representing a depressed mood, icon 112 and character string 122 representing a gloomy mood, icon 113 and character string 123 representing a neutral mood, icon 114 and character string 124 representing a satisfied mood, and icon 115 and character string 125 representing a joyful mood. The user can select any one of icons 111 to 115 by touching input screen 100 or by other means to input the first parameter representing the user's subjective mood.

When the user inputs the first parameter, second parameter input screen 200 illustrated in FIG. 3 is displayed on user interface 11 of information terminal 1. This second parameter input screen 200 displays character string 201 representing a question to the user, “How energetic are you now?”, a plurality of (five in this case) icons 211 to 215 representing the second parameter, and a plurality of character strings 221 to 225 describing the plurality of icons 211 to 215, respectively. Each of the plurality of character strings 221 to 225 is a value representing the energy degree as a percentage. In the lower portion of input screen 200, the icons and character strings are displayed in order from the left, with icon 211 and character string 221 representing that the user is not energetic at all, icon 212 and character string 222 representing that the user is not very energetic, icon 213 and character string 223 representing that the user is neutral, icon 214 and character string 224 representing that the user is somewhat energetic, and icon 215 and character string 225 representing that the user is very energetic. The user can select any one of the icons 211 to 215 by touching input screen 200 or by other means to input the second parameter indicating the user's subjective excitement degree.

When the user inputs the second parameter, third parameter input screen 300 illustrated in FIG. 4 is displayed on user interface 11 of information terminal 1. This input screen 300 displays character string 301 representing a question to the user, “How relaxed are you now?”, a plurality of (five in this case) icons 311 to 315 representing the third parameter, and a plurality of character strings 321 to 325 describing the plurality of icons 311 to 315, respectively. In the lower part of input screen 300, the icons and character strings are displayed in order from the left, with icon 311 and character string 321 representing that the user is not relaxed at all, icon 312 and character string 322 representing that the user is not very relaxed, icon 313 and character string 323 representing that the user is neutral, icon 314 and character string 324 representing that the user is somewhat relaxed, and icon 315 and character string 325 representing that the user is very relaxed. The user can select any one of icons 311 to 315 by touching input screen 300 or by other means to input the third parameter indicating the user's subjective relaxation degree.

In the embodiment, first parameter input screen 100, second parameter input screen 200, and third parameter input screen 300 are displayed in this order on user interface 11 of information terminal 1. However, the order in which input screens 100 to 300 are displayed is not limited to this order. For example, third parameter input screen 300, second parameter input screen 200, and first parameter input screen 100 may be displayed in this order on user interface 11 of information terminal 1.

In the embodiment, each of the first parameter, the second parameter, and the third parameter is represented by five scales, but the present disclosure is not limited thereto. For example, at least one of the first parameter, the second parameter, and the third parameter may be represented on scales of fewer than five or more than five.

### [1-5. Estimation of User's Emotion]

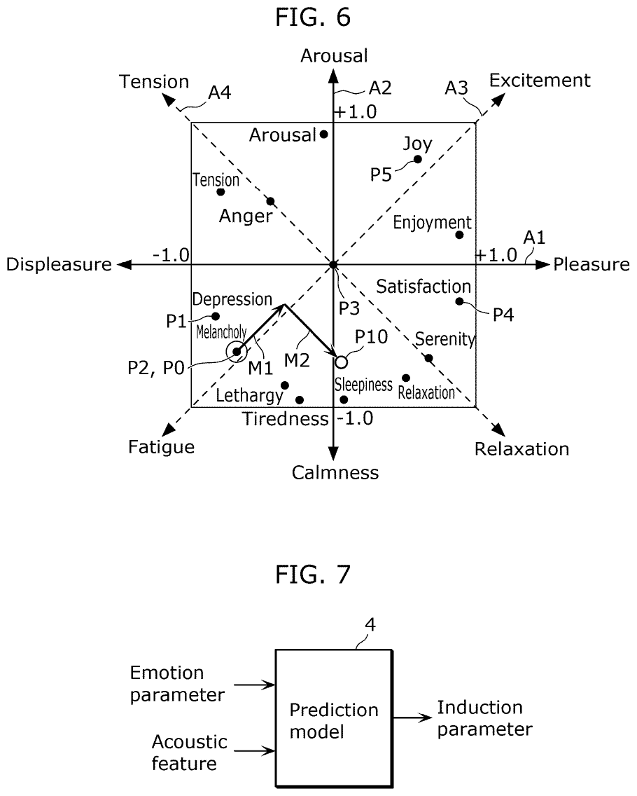

The information processing for estimating the emotion parameter by CPU 13 of emotion estimation system 10 will be described below with reference to FIGS. 5 and 6. FIG. 5 is a diagram illustrating an example of an affect grid according to the embodiment. FIG. 5 is an explanatory diagram of the estimation of the user's emotion by emotion estimation system 10 according to the embodiment. FIGS. 5 and 6 are diagrams both illustrating a two-dimensional orthogonal coordinate system based on the affect Grid method for evaluating an emotion parameter indicating the user's emotion in two dimensional coordinates. For the Affect Grid method, see Russell, J. A., Weiss, A., & Mendelsohn, G. A. (1989). Affect grid: A single-item scale of pleasure and arousal. Journal of Personality and Social Psychology, 57 (3), 493.

As illustrated in FIGS. 5 and 6, the emotion parameter is represented by the coordinates of a plane (affect grid) defined by first axis A1 indicating the comfort degree and second axis A2 indicating the arousal degree. On first axis A1, the comfort degree is shown by a value from −1.0 (displeasure) to +1.0 (pleasure). On second axis A2, the arousal degree is shown by a value from −1.0 (calmness) to +1.0 (arousal). Note that the numerical value of the comfort degree and the numerical value of the arousal degree are both numerical values neutralized within a range of ±1.0, and are not intended to be limited to these numerical values.

In the affect grid of the embodiment, third axis A3 indicating the excitement degree and fourth axis A4 indicating the relaxation degree are further defined. Third axis A3 is an axis obtained by rotating first axis A1 by first angle θ1 with respect to the origin of the plane (affect grid). Here, first angle θ1 is 45 degrees, and third axis A3 is an axis obtained by rotating first axis A1 counterclockwise with respect to the origin. On third axis A3, the excitement degree is shown such that the higher the degree to which the user feels excited, the larger the positive value, and that the higher the degree to which the user feels fatigue, the larger the negative value. Note that first angle θ1 is not limited to 45 degrees, and needs only to be an angle capable of indicating the excitement degree.

Fourth axis A4 is an axis obtained by rotating second axis A2 by second angle θ2 with respect to the origin of the plane (affect grid). Here, second angle θ2 is 45 degrees, and fourth axis A4 is an axis obtained by rotating second axis A2 counterclockwise with respect to the origin. On fourth axis A4, the relaxation degree is shown such that the higher to which the user is relaxed, the larger the user's relaxation degree, and that the higher the degree to which the user feels tense, the larger the negative value. Note that second angle θ2 is not limited to 45 degrees, and needs only to be an angle capable of indicating the relaxation degree.

CPU 13 of emotion estimation system 10 first determines starting point P0 (see FIG. 6) on the plane (affect grid) based on the first parameter. Specifically, when the user selects icon 111 on input screen 100, that is, when the user inputs a first parameter indicating that the user is in a depressed mood, CPU 13 determines point P1 as starting point P0. When the user selects icon 112 on input screen 100, that is, when the user inputs a first parameter indicating that the user is in a gloomy mood, CPU 13 determines point P2 as starting point P0. When the user selects icon 113 on input screen 100, that is, when the user inputs a first parameter indicating that the user is in a neutral mood, CPU 13 determines point P3 as starting point P0. Point P3 is the origin of the affect grid. When the user selects icon 114 on input screen 100, that is, when the user inputs the first parameter indicating that the user is in a satisfied mood, CPU 13 determines point P4 as starting point P0. When the user selects icon 115 on input screen 100, that is, when the user inputs a first parameter indicating that the user is in a joyful mood, CPU 13 determines point P5 as starting point P0.

Next, CPU 13 determines first movement amount M1 (see FIG. 6). First movement amount M1 is a movement amount based on the second parameter along third axis A3. In other words, first movement amount M1 is represented by a vector parallel to third axis A3. Specifically, when the user selects icon 211 on input screen 200, that is, when the user inputs a second parameter indicating that the user is not energetic at all, CPU 13 determines first movement amount M1 to be a vector in a negative direction (leftward and downward in FIG. 6). When the user selects icon 212 on input screen 200, that is, when the user inputs a second parameter indicating that the user is not very energetic, CPU 13 determines first movement amount M1 to be a vector in the negative direction. The movement amount of this vector is smaller than that of the vector when the user is not energetic at all. When the user selects icon 213 on input screen 200, that is, when the user inputs a second parameter indicating that the user is neutral, CPU 13 determines first movement amount M1 to be zero. When the user selects icon 214 on input screen 200, that is, when the user inputs a second parameter indicating that the user is somewhat energetic, CPU 13 determines first movement amount M1 to be a vector in the positive direction (rightward and upward in FIG. 6). When the user selects icon 215 on input screen 200, that is, when the user inputs a second parameter indicating that the user is very energetic, CPU 13 determines first movement amount M1 to be a vector in the positive direction. The movement amount of this vector is larger than that of the vector when the user is somewhat energetic.

Next, CPU 13 determines second movement amount M2 (see FIG. 6). Second movement amount M2 is a movement amount based on the third parameter along fourth axis A4. In other words, second movement amount M2 is represented by a vector parallel to fourth axis A4. Specifically, when the user selects icon 311 on input screen 300, that is, when the user inputs a third parameter indicating that the user is not relaxed at all, CPU 13 determines second movement amount M2 to be a vector in the negative direction (leftward and upward in FIG. 6). When the user selects icon 312 on input screen 300, that is, when the user inputs a third parameter indicating that the user is not very relaxed, CPU 13 determines second movement amount M2 to be a vector in the negative direction. The movement amount of this vector is smaller than that of the vector when the user is not relaxed at all. When the user selects icon 313 on input screen 300, that is, when the user inputs a third parameter indicating that the user is neutral, CPU 13 determines second movement amount M2 to be zero. When the user selects icon 314 on input screen 300, that is, when the user inputs a third parameter indicating that the user is somewhat relaxed, CPU 13 determines second movement amount M2 to be a vector in the positive direction (rightward and downward in FIG. 6). When the user selects icon 315 on input screen 300, that is, when the user inputs a third parameter indicating that the user is very relaxed, CPU 13 determines second movement amount M2 to be a vector in the positive direction. The movement amount of this vector is larger than that of the vector when the user is somewhat relaxed.

Then, CPU 13 estimates emotion parameter P10 by moving starting point P0 according to first movement amount M1 and second movement amount M2. That is, CPU 13 moves starting point P0 by the vector indicated by first movement amount M1 and further by the vector indicated by second movement amount M2 in the affect grid, and estimates the coordinates after the movement as emotion parameter P10. FIG. 6 illustrates an example of emotion parameter P10 when the user selects icon 112 on input screen 100 (that is, the user inputs the first parameter indicating that the user is in a gloomy mood), the user selects icon 214 on input screen 200 (that is, the user inputs the second parameter indicating that the user is somewhat energetic), and the user selects icon 314 on input screen 300 (that is, the user inputs the third parameter indicating that the user is somewhat relaxed).

As described above, the emotion parameter indicating the user's emotion is represented by the comfort degree and the arousal degree. Then, emotion estimation system 10 according to the embodiment can estimate the user's emotion by determining the comfort degree and the arousal degree based on the user's subjective evaluation of the user's emotion, that is, based on the first parameter indicating the user's subjective mood, the second parameter indicating the user's subjective excitement degree, and the third parameter indicating the user's subjective relaxation degree. Therefore, emotion estimation system 10 does not require obtaining the user's biometric data as in the technique disclosed in Patent Document 1, thus eliminating the need to separately prepare means for obtaining the user's biometric data. Further, emotion estimation system 10 can estimate the user's emotion based on the user's three subjective evaluations, thus eliminating the need for the user to respond to a large number of inquiries. Accordingly, emotion estimation system 10 has the advantage of facilitating the estimation of the user's emotion by a relatively simple method.

### [1-6. Determination of Sound Content]

In the following, the information processing for determining the sound content by CPU 22 of content determination system 20 will be described. CPU 22 determines the sound content so that the emotion parameter estimated by emotion estimation system 10 changes to an induction parameter indicating an emotion that the user is induced to feel. In other words, CPU 22 determines the sound content so that the user's emotion before the sound content is played back is changed to a predetermined emotion by playing back the sound content. The induction parameter referred to here is a type of emotion parameter, and is a parameter indicating an emotion that the user is intended to feel by listening to the sound content.

In the embodiment, CPU 22 implements information processing to determine the sound content by referring to database 25. Then, database 25 is constructed in advance using machine-trained prediction model 4 illustrated in FIG. 7. FIG. 7 is an explanatory diagram of prediction model 4 used in content determination system 20 according to the embodiment. Prediction model 4 is a neural network machine-trained through supervised learning so that an emotion parameter after the sound content playback is output using, as inputs, an acoustic feature an emotion parameter before the sound content playback. In other words, prediction model 4 is a model that evaluates what emotion a user who is feeling a certain emotion will be induced to feel when sound content is played back to the user.

Here, the acoustic feature is the physical feature of the sound signal extracted from the sound content. For example, the acoustic feature may include tempo (the speed of the sound content), beat whiteness (the ratio of occurrence frequencies between sounds that contribute to beat formation and those that do not), spectral variability (the degree of spectral change between frames of a predetermined time length), and an average number of sounds pronounced (the frequency of sounds pronounced in the sound content). The acoustic feature may also include, for example, features such as mel-frequency cepstral coefficients (MFCCs), which are spectra representing timbre in consideration of human auditory characteristics, sound chords, and contrast in energy distribution in the frequency domain. One or more of these acoustic features are used when prediction model 4 is machine-trained.

Prediction model 4 is machine-trained using a large number of learning datasets prepared in advance. The learning dataset includes an emotion parameter and an acoustic feature as input data, and correct answer data. The learning dataset can be generated by, for example, causing a subject who has input the first parameter, the second parameter, and the third parameter to listen to the sound content and then input the first parameter, the second parameter, and the third parameter again. In other words, the learning dataset includes an emotion parameter based on the first parameter, second parameter, and third parameter input by the subject before listening to the sound content, an acoustic feature extracted from the sound content to be listened to by the subject, and an emotion parameter as correct answer data based on the first parameter, second parameter, and third parameter input by the subject after listening to the sound content.

Here, the change in emotion caused by the subject listening to the sound content can vary depending on the time period during which the subject listens to the sound content. That is, for example, due to the fatigue degree of the subject or the amount of sunlight to which the subject is directly or indirectly exposed, the change in the emotion of the subject may vary even when the subject listens to the same sound content. Therefore, in the embodiment, three prediction models 4 corresponding to three time periods of morning, afternoon, and evening are prepared.

Next, the construction of database 25 will be described. First, an acoustic feature is extracted from any given sound content. Then, the extracted acoustic feature and the emotion parameter before the sound content is listened to are input to machine-trained prediction model 4, thereby obtaining an emotion parameter after the sound content is listened to, outputted from prediction model 4. An operation similar to the above is performed on all the emotion parameters while the acoustic feature input to prediction model 4 remains fixed. Thus, for any given sound content, it is possible to obtain a dataset that links: an identifier of the sound content corresponding to the extracted acoustic feature (for example, the song title of the sound content); an emotion parameter before the sound content is listened to; an emotion parameter after the sound content is listened to; and a classification probability. Here, the classification probability means a probability that prediction model 4 classifies the sound content into the emotion parameter after the sound content is listened to. By performing the above operation on all the prepared sound contents and all prediction models 4, it is possible to obtain a dataset for each of all the sound contents, that is, to construct database 25.

Then, CPU 22 executes information processing for determining sound content, using database 25 constructed as described above. Specifically, CPU 22 searches database 25 for sound content that matches the combination of the emotion parameter (the emotion parameter before the sound content is listened to) and the induction parameter (the emotion parameter after the sound content is listened to). When there is no sound content that matches the combination of the emotion parameter and the induction parameter, CPU 22 searches database 25 for the sound content closest to the combination. Then, CPU 22 determines the sound content by preferentially selecting a sound content with a high classification probability among the searched sound contents, and generates a playlist including the determined sound content.

### 2. Operation

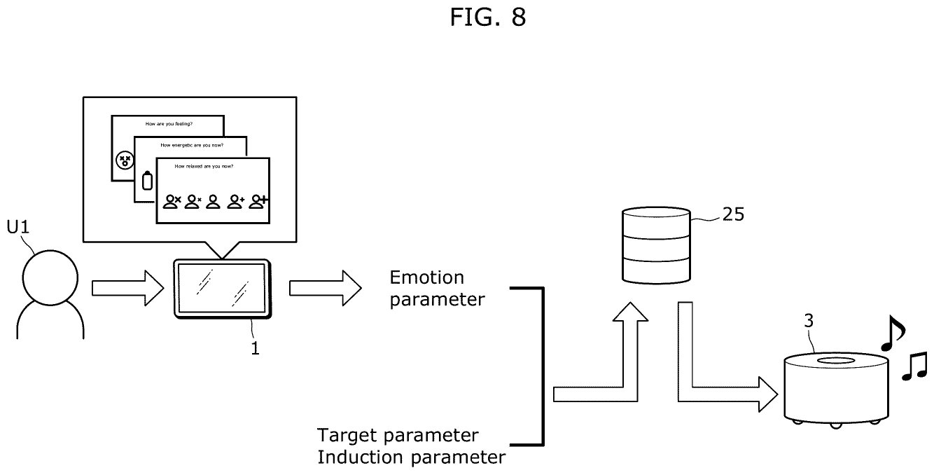

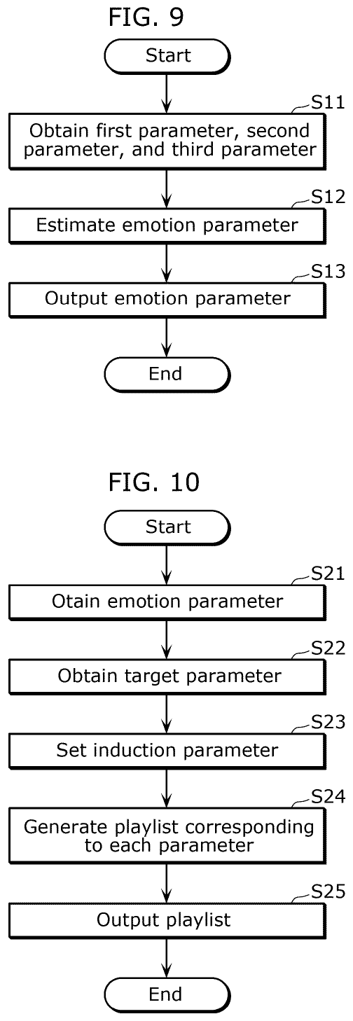

An example of the operations of emotion estimation system 10 and content determination system 20 according to the embodiment will be described below with reference to FIGS. 8, 9, and 10. FIG. 8 is a schematic diagram illustrating an example of the operations of emotion estimation system 10 and content determination system 20 according to the embodiment. FIG. 9 is a flowchart illustrating an example of the operation of emotion estimation system 10 according to the embodiment. FIG. 10 is a flowchart illustrating an example of the operation of content determination system 20 according to the embodiment. In the following, a description will be given assuming that there are a plurality of users U1 in the office.

First, an example of the operation of emotion estimation system 10 will be described. Each user U1 inputs the first parameter, the second parameter, and the third parameter through user interface 11 of information terminal 1 used by user U1. Thus, user interface 11 obtains the first parameter, the second parameter, and the third parameter (S11). Next, CPU 13 of information terminal 1 estimates an emotion parameter indicating the emotion of user U1 based on the first parameter, second parameter, and third parameter obtained by user interface 11 (S12). Then, communication interface 12 of information terminal 1 transmits a signal including the emotion parameter estimated by CPU 13 to server 2 via network N1 to output the emotion parameter (S13). As a result, the emotion parameter estimated by information terminal 1 of each user U1 is output to server 2.

Next, an example of the operation of content determination system 20 will be described. Communication interface 21 (input interface 21A) of server 2 receives the signal transmitted from information terminal 1 to obtain the emotion parameter (S21). Here, communication interface 21 obtains the emotion parameter of each user U1. Communication interface 21 also receives the signal transmitted from the information terminal of the administrator to obtain the target parameter (S22). Similarly to the induction parameter, the target parameter referred to here is a parameter indicating an emotion that each user U1 is intended to feel by listening to the sound content, but is a parameter different from the induction parameter. That is, the target parameter is a parameter indicating an emotion that each user U1 is intended to ultimately feel by listening to the sound content.

In the embodiment, the emotional tendency of each target user U1 varies among three time periods of morning, afternoon, and evening. Specifically, in the morning time period, an emotion with relatively high excitement degree and arousal degree (that is, an emotion shown in an area above third axis A3 in the first quadrant of the affect grid illustrated in FIG. 5) is set as a target. In the afternoon time period, an emotion with relatively high excitement degree and comfort degree (that is, an emotion shown in a region below third axis A3 in the first quadrant of the affect grid illustrated in FIG. 5) is set as a target. In the evening time period, an emotion with a relatively high relaxation degree (that is, an emotion shown in the fourth quadrant of the affect grid illustrated in FIG. 5) is set as a target. Therefore, here, communication interface 21 obtains the target parameters for the respective time periods of morning, afternoon, and evening. Step S22 may be performed prior to Step S21.

Next, CPU 22 of server 2 executes information processing for determining the sound content based on the obtained emotion parameter and target parameter. In this information processing, the representative value of the emotion parameter of each user U1 is used. For example, the representative value is a moving average value of the emotion parameter of each user U1. The moving average value may be calculated by weighting according to the stay time of each user U1 in the office. For example, the shorter the stay time of user U1, the greater the weighting may be made, and the longer the stay time of user U1, the smaller the weighting may be made.

CPU 22 sets an induction parameter based on the obtained emotion parameter and target parameter (S23). In step S23, CPU 22 obtains the current time and selects one target parameter from the three target parameters corresponding to the three time periods of morning, afternoon, and evening, respectively, based on the current time. Next, CPU 22 generates a playlist corresponding to the set induction parameter and target parameter (S24). Then, communication interface 21 (output interface 21B) transmits a signal including the playlist generated by CPU 22 to playback system 3 via network N1 to outputs the playlist (S25).

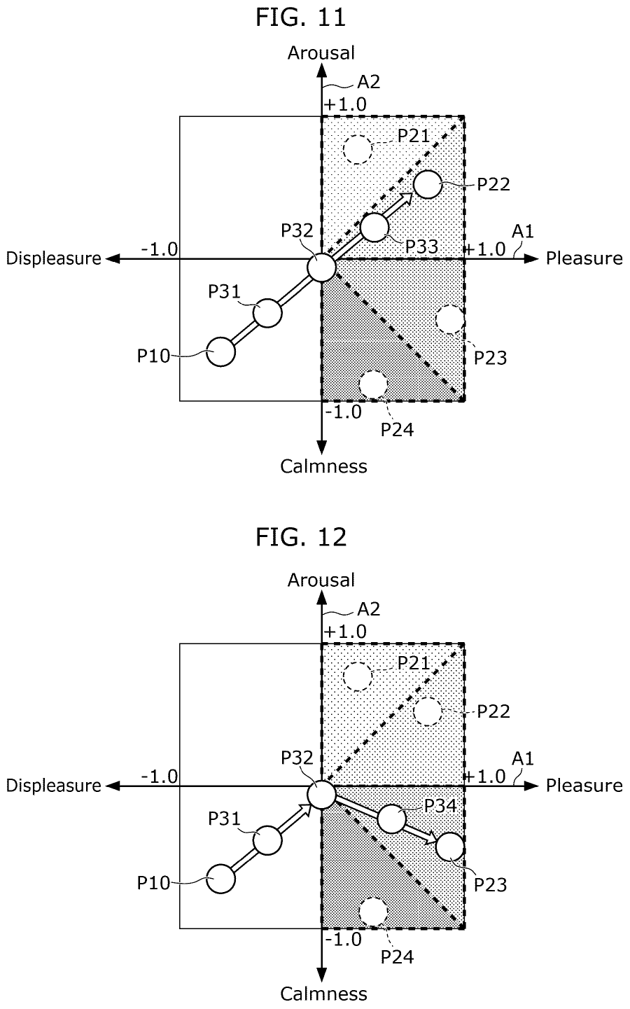

Here, the setting of the induction parameter and the generation of the playlist will be described with reference to FIG. 11. FIG. 11 is an explanatory diagram of an example of the playlist generation in content determination system 20 according to the embodiment. FIG. 11 illustrates the affect grid similarly to FIG. 5. In FIG. 11, third axis A3 and fourth axis A4 are omitted. FIG. 11 illustrates target parameter P21 for the morning time period, target parameter P22 for the afternoon time period, and target parameters P23, P24 for the evening time period. In the example illustrated in FIG. 11, the time period is afternoon, and CPU 22 uses target parameter P22.

First, CPU 22 calculates a distance (that is, a difference) between emotion parameter P10 and target parameter P22 on the affect grid. Then, based on the calculated distance, CPU 22 sets the induction parameter so that emotion parameter P10 approaches target parameter P22. For example, CPU 22 sets the induction parameter so that the calculated distance is divided into equal intervals. In the example illustrated in FIG. 11, CPU 22 sets three induction parameters P31 to P33. When the calculated distance is shorter than a threshold, that is, when the emotion parameter and the target parameter are close to each other, CPU 22 need not set the induction parameter.

Next, CPU 22 generates a playlist corresponding to each of the induction parameter and the target parameter. Here, CPU 22 generates a playlist corresponding to each of three induction parameters P31 to P33 and a playlist corresponding to the target parameter.

For example, when generating a playlist corresponding to induction parameter P31, CPU 22 determines the sound content by searching database 25 for the sound content that matches the combination of emotion parameter P10 and induction parameter P31, and generates a playlist including the determined sound content.

For example, when generating a playlist corresponding to induction parameter P32, CPU 22 determines the sound content by using induction parameter P31 as the emotion parameter and searching database 25 for the sound content that matches the combination of the emotion parameter and induction parameter P32, and generates a playlist including the determined sound content. For example, when generating a playlist corresponding to induction parameter P33, CPU 22 determines the sound content by using induction parameter P32 as the emotion parameter and searching database 25 for the sound content that matches the combination of the emotion parameter and induction parameter P33, and generates a playlist including the determined sound content.

For example, when generating a playlist corresponding to target parameter P22, CPU 22 determines the sound content by using induction parameter P33 as the emotion parameter and target parameter P22 as the induction parameter and searching database 25 for the sound content that matches the combination of the emotion parameter and the induction parameter, and generates a playlist including the determined sound content.

Playback system 3, having received the signal that includes the playlist, plays back the sound content according to the obtained playlist. For example, when CPU 22 of content determination system 20 generates a playlist in accordance with the example illustrated in FIG. 11, playback system 3 first plays back the sound content for a predetermined time (for example, 30 minutes) in accordance with the playlist corresponding to induction parameter P31. Thereafter, playback system 3 plays back the sound content for respective predetermined times in accordance with the playlist corresponding to induction parameter P32, the playlist corresponding to induction parameter P33, and the playlist corresponding to target parameter P22.

If playback system 3 plays back the sound content in accordance with the playlist corresponding to target parameter P22 without playing back the sound content in accordance with each of the playlists corresponding to induction parameters P31 to P33, the following problem may occur. That is, since the emotion represented by target parameter P22 is greatly different from the emotion represented by emotion parameter P10, even if the sound content is played back in accordance with the playlist corresponding to target parameter P22, user U1 just feels displeased, and the effect of inducing the emotion of user U1 to the emotion represented by target parameter P22 cannot be expected.

On the other hand, as described above, playback system 3 plays back the sound content in accordance with each of the playlists corresponding to induction parameters P31 to P33, so that the emotion of user U1 can be gradually induced from the emotion represented by emotion parameter P10 to the emotion represented by target parameter P22.

Meanwhile, the time period may change during the playback of the sound content by playback system 3. In such a case, CPU 22 of content determination system 20 resets the induction parameter and the target parameter and regenerates the playlist based on the reset induction parameter and target parameter.

FIG. 12 is an explanatory diagram of an example of the playlist regeneration in content determination system 20 according to the embodiment. FIG. 12 illustrates an affect grid similarly to FIG. 11, and third axis A3 and fourth axis A4 are omitted. Similarly to FIG. 11, FIG. 12 illustrates target parameter P21 in the morning time period, target parameter P22 in the afternoon time period, and target parameters P23, P24 in the evening time period. In the example illustrated in FIG. 12, it is assumed that in the afternoon time period, one or more induction parameters and a playlist corresponding to the one or more induction parameters are generated based on target parameter P22 for the afternoon time period. In the example illustrated in FIG. 12, it is assumed that the time period has changed from afternoon to evening when playback system 3 is playing back the sound content in accordance with the playlist corresponding to induction parameter P32.

In the example illustrated in FIG. 12, after the completion of the playback of the playlist (here, the playlist corresponding to induction parameter P32) that was being played back at the time when the time period changed from afternoon to evening, CPU 22 resets the induction parameter and the target parameter and regenerates the playlist, and starts the playback of the playlist regenerated in playback system 3.

Specifically, CPU 22 first resets the target parameter from target parameter P22 to target parameter P23. At the time when the time period changes from afternoon to evening, the emotion of user U1 is estimated to be between the emotion represented by induction parameter P31 and the emotion represented by induction parameter P32. Then, when the playback of the playlist corresponding to induction parameter P32 being played back at that time is completed, the emotion of user U1 is estimated to be induced to the emotion represented by induction parameter P32. Then, CPU 22 calculates a distance (that is, a difference) between the emotion parameter and new target parameter P23, using induction parameter P32 as the emotion parameter serving as a new starting point. Then, based on the calculated distance, CPU 22 resets the new induction parameter so that the new emotion parameter approaches new target parameter P23. In the example illustrated in FIG. 12, CPU 22 resets induction parameter P34 as a new induction parameter.

CPU 22 regenerates the playlist corresponding to reset induction parameter P34 and the playlist corresponding to reset target parameter P23. Then, communication interface 21 (output interface 21B) transmits a signal including the playlist regenerated by CPU 22 to playback system 3 via network N1. As a result, after the sound content is played back in accordance with the playlist corresponding to induction parameter P32, playback system 3 plays back the sound content for respective predetermined times in accordance with the playlist corresponding to reset induction parameter P34 and the playlist corresponding to reset target parameter P23. This enables the emotion of user U1 to be induced to the emotion represented by the target parameter corresponding to the time period after the change.

In the above operation, at the time when the time period changes from afternoon to evening, CPU 22 may immediately terminate the playback of the playlist (here, the playlist corresponding to induction parameter P32) currently being played back, reset the induction parameter and the target parameter, regenerate the playlist, and cause playback system 3 to start the playback of the regenerated playlist. That is, in this case, similarly to the example illustrated in FIG. 12, CPU 22 resets the new induction parameter and regenerates the playlist corresponding to the new induction parameter, using induction parameter P32 as the emotion parameter serving as a new starting point. However, in this case, the playback of the playlist corresponding to the new induction parameter is not started at the time when the playback of the current playlist (the playlist corresponding to induction parameter P32) is completed, but is started at the time when the time period changes from afternoon to evening.

In the above operation, at the time when the time period changes from afternoon to evening, CPU 22 may immediately terminate the playback of the playlist (here, the playlist corresponding to induction parameter P32) currently being played back. Then, CPU 22 may reset the induction parameter and the target parameter and regenerate the playlist, using the induction parameter (here, induction parameter P31) corresponding to the playlist immediately preceding the playlist being played back at that time as the emotion parameter serving as a new starting point. That is, in this case, CPU 22 resets the new induction parameter and regenerates the playlist corresponding to the new induction parameter, using induction parameter P31 as the emotion parameter serving as a new starting point. In this case, the playback of the playlist corresponding to the new induction parameter is started at the time when the time period changes from afternoon to evening.

### 3. Effects, etc.

As described above, an emotion estimation method according to a first aspect executed by a computer such as CPU 13 includes: obtaining a first parameter indicating the user's subjective mood, a second parameter indicating the user's subjective excitement degree, and a third parameter indicating the user's subjective relaxation degree (S11); estimating an emotion parameter indicating the user's emotion based on the obtained first parameter, second parameter, and third parameter (S12); and outputting information related to the emotion parameter (S13).

Therefore, such an emotion estimation method does not require obtaining the user's biometric data as in the technique disclosed in Patent Document 1, thus eliminating the need to separately prepare means for obtaining the user's biometric data. Further, such an emotion estimation method can estimate the user's emotion based on the user's three subjective evaluations, thus eliminating the need for the user to respond to a large number of inquiries. Accordingly, such an emotion estimation method has the advantage of facilitating the estimation of the user's emotion by a relatively simple method.

For example, in an emotion estimation method according to a second aspect, in the first aspect, the emotion parameter is represented as coordinates of a plane defined by first axis A1 indicating the comfort degree and second axis A2 indicating the arousal degree. In an emotion estimation method, starting point P0 on the plane is determined based on the first parameter, and the emotion parameter is estimated by moving starting point P0 according to first movement amount M1 and second movement amount M2. First movement amount M1 is based on the second parameter along third axis A3 obtained by rotating first axis A1 by first angle θ1 with respect to the origin of the plane. Second movement amount M2 is based on a third parameter along fourth axis A4 obtained by rotating second axis A2 by second angle θ2 with respect to the origin.

Such an emotion estimation method has the advantage of facilitating the quantitative estimation of the user's emotion by representing the user's emotion in terms of the coordinates of the plane.

For example, in an emotion estimation method according to a third aspect, in the second aspect, both first angle θ1 and second angle θ2 are 45 degrees. Third axis A3 is an axis indicating the excitement degree, and fourth axis A4 is an axis indicating the relaxation degree.

Such an emotion estimation method has the advantage of facilitating the reflection of the second parameter and the third parameter in coordinates of the plane when the user's emotion is represented by the coordinates.

For example, a content determination method according to a fourth aspect executed by a computer such as CPU 22 includes: obtaining the emotion parameter estimated by the emotion estimation method according to any one of the first to third aspects (S21); determining content to be provided to the user, based on the obtained emotion parameter (S24); and outputting information related to the determined content (S25).

Such a content determination method has the advantage of facilitating, for example, the provision of content to induce the user to feel a predetermined emotion by providing the user with content corresponding to the estimated user's emotion.

For example, a content determination method according to a fifth aspect further includes, in the fourth aspect: obtaining a target parameter indicating a target emotion for the user (S22); and determining the content based on a difference between the emotion parameter and the target parameter (S24)

Such a content determination method has the advantage of making it easier to provide the user with content that induces the user to feel a target emotion.

For example, a content determination method according to a sixth aspect further includes, in the fifth aspect: setting, based on the difference, an induction parameter indicating an emotion that the user is induced to feel, to cause the emotion parameter to approach the target parameter (S23); and determining the content based on the induction parameter (S24).

Such a content determination method has the advantage of facilitating the provision of content, which is expected to be even more effective in inducing the user to feel the target emotion, to the user.

For example, a recording medium according to a seventh aspect is a non-transitory computer-readable recording medium having recorded thereon a computer program for causing a computer such as CPU 13 to execute the emotion estimation method according to any one of the first to third aspects.

Such a recording medium can produce an effect similar to the above emotion estimation method.

For example, a recording medium according to an eighth aspect is a non-transitory computer-readable recording medium having recorded thereon a computer program for causing a computer such as CPU 22 to execute the content determination method according to any one of the fourth to sixth aspects.

Such a recording medium can produce an effect similar to the above content determination method.

For example, emotion estimation system 10 according to a ninth aspect includes user interface 11, CPU 13, and communication interface 12. User interface 11 obtains a first parameter indicating the user's subjective mood, a second parameter indicating the user's subjective excitement degree, and a third parameter indicating the user's subjective relaxation degree. CPU 13 estimates an emotion parameter indicating the user's emotion, based on the obtained first parameter, second parameter, and third parameter. Communication interface 12 outputs information related to the emotion parameter. User interface 11 is an example of an input interface. CPU 13 is an example of a signal processing circuit. Communication interface 12 is an example of an output interface.

Such emotion estimation system 10 can produce an effect similar to the above emotion estimation method.

For example, content determination system 20 according to a tenth aspect includes input interface 21A, CPU 22, and output interface 21B. Input interface 21A obtains the emotion parameter estimated by emotion estimation system 10 according to the ninth aspect. CPU 22 determines the content to be provided to the user, based on the obtained emotion parameter. Output interface 21B outputs information related to the determined content. CPU 22 is an example of a signal processing circuit.

Such content determination system 20 can produce an effect similar to the above content determination method.

### 4. Other Embodiments

Although the embodiment has been described above, the present disclosure is not limited to the above embodiment.

In the above embodiment, emotion estimation system 10 is implemented by information terminal 1, and content determination system 20 is implemented by server 2, but the present disclosure is not limited thereto. For example, both emotion estimation system 10 and content determination system 20 may be implemented by information terminal 1. In this case, server 2 is not needed. For example, emotion estimation system 10 may be implemented by server 2. In this case, input interface 21A of server 2 receives a signal including the first parameter, second parameter, and third parameter input by information terminal 1 to obtain the respective parameters.

In the above embodiment, emotion estimation system 10, content determination system 20, and playback system 3 are implemented by devices independent of each other, but the present disclosure is not limited thereto. For example, server 2 and playback system 3 may be implemented by one device. Further, for example, all of emotion estimation system 10, content determination system 20, and playback system 3 may be implemented by one device. In this case, the one device is installed, for example, in an office.

In the above embodiment, playback system 3 reads the sound content stored in storage device 34 and plays back the sound content using loudspeaker 36, but the present disclosure is not limited thereto. For example, playback system 3 may play back the sound content through so-called streaming in which the sound content transmitted from server 2 via network N1 is received and played back by loudspeaker 36. In this case, playback system 3 need not include storage device 34. In this case, server 2 needs only to be provided with a storage device for storing a plurality of sound contents. Note that playback system 3 may receive sound content transmitted from a server different from server 2 and operated by a music distributor.

In the above embodiment, playback system 3 is configured to play back the sound content determined by content determination system 20 from a predetermined place in the office to the user, but the present disclosure is not limited thereto. For example, playback system 3 may be implemented by information terminal 1. In this case, the user may listen to sound content played back from the built-in loudspeaker of information terminal 1, or may listen to sound content played back via earphones connected to information terminal 1. For example, information terminal 1 may receive the sound content transmitted from playback system 3 via network N1, and play back the sound content through streaming in which the sound content is played back by a loudspeaker built in information terminal 1.

In the above embodiment, database 25 is constructed using prediction model 4 that has been machine-trained, but the present disclosure is not limited thereto. For example, database 25 may be constructed on a rule basis without using machine-trained prediction model 4.

In the above embodiment, prediction model 4 may be the following prediction model. That is, the prediction model may be a model using the user's emotional parameter before the sound content is listened to and the user's emotional parameter after the sound content is listened to as inputs and an acoustic feature as an output. In this case, when the current emotion parameter and the induction parameter to which the user is intended to be induced are input to the machine-trained prediction model, an acoustic feature is output. Thus, from the database that maps sound content to an acoustic feature, it is possible to select the sound content that has the closest acoustic feature to the acoustic feature output by the prediction model.

In the above embodiment, when the time period changes during the playback process of the sound content, content determination system 20 changes the playlist so that the user is induced to feel the target parameter corresponding to the changed time period. However, the present disclosure is not limited thereto. For example, content determination system 20 may maintain the initially determined playlist even if the time period changes during the playback process of the content.

In the above embodiment, communication interface 21 of server 2 serves as both input interface 21A and output interface 21B, but the present disclosure is not limited thereto. For example, input interface 21A and output interface 21B may be interfaces different from each other.

In the above embodiment, the emotion estimation system is implemented by a single device, but may be implemented by a plurality of devices. When emotion estimation system 10 is implemented by a plurality of devices, the functional components included in the emotion estimation system may be distributed to the plurality of devices in any manner. Similarly, in the above embodiment, the content determination system is implemented by a single device, but may be implemented by a plurality of devices. When the content determination system is implemented by a plurality of devices, the functional components included in the content determination system may be distributed to the plurality of devices in any manner.

The communication method between the devices in the above embodiment is not particularly limited. In the above embodiment, when two devices perform communication, a relay device (not illustrated) may be interposed between the two devices.

The order of the processing described in the above embodiment is an example. The order of the plurality of pieces of processing may be changed, and the plurality of pieces of processing may be executed in parallel. Further, the processing performed by a specific processor may be performed by another processor. A part of the digital signal processing described in the above embodiment may be implemented by analog signal processing.

In the above embodiment, each of the components may be implemented by executing a software program suitable for each of the components. Each of the components may be implemented by a program execution unit, such as a CPU or processor, reading and executing a software program recorded in a recording medium, such as a hard disk or a semiconductor memory.

Each of the components may be implemented by hardware. For example, each of the components may be a circuit (or an integrated circuit). These circuits may constitute one circuit as a whole or may be separate circuits. These circuits may each be a general-purpose circuit or a dedicated circuit.

General or specific aspects of the present disclosure may also be realized by a system, device, method, integrated circuit, computer program, or a computer-readable storage medium such as a compact disc read-only memory (CD-ROM). The general or specific aspect of the present disclosure may also be realized by any combination of the system, device, method, integrated circuit, computer program, and recording medium. For example, the present disclosure may be executed as an emotion estimation method executed by a computer, or may be implemented as a program for causing a computer to execute such an emotion estimation method. Similarly, the present disclosure may be executed as a content determination method executed by a computer, or may be implemented as a program for causing a computer to execute such a content determination method. The present disclosure may be realized as a computer-readable non-transitory recording medium in which such a program is recorded. Here, the program includes an application program for causing a general-purpose information terminal to function as the information terminal of the above embodiment.

In addition, the present disclosure also includes forms obtained by application of various modifications conceivable by one skilled in the art to each of the embodiments, or forms realized by any combination of the components and functions in each of the embodiments, without departing from the spirit of the present disclosure.

### INDUSTRIAL APPLICABILITY

The emotion estimation method of the present disclosure can estimate the user's emotion based on the user's subjective mood.

1. An emotion estimation method comprising:
obtaining a first parameter indicating a subjective mood of a user, a second parameter indicating a subjective excitement degree of the user, and a third parameter indicating a subjective relaxation degree of the user;
estimating an emotion parameter indicating an emotion of the user based on the first parameter obtained, the second parameter obtained, and the third parameter obtained; and
outputting information related to the emotion parameter.

2. The emotion estimation method according to claim 1,
wherein the emotion parameter is represented as coordinates of a plane defined by a first axis indicating a comfort degree and a second axis indicating an arousal degree,
a starting point on the plane is determined based on the first parameter, and
the emotion parameter is estimated by moving the starting point according to a first movement amount and a second movement amount, the first movement amount being based on the second parameter along a third axis obtained by rotating the first axis by a first angle with respect to an origin of the plane, the second movement amount being based on the third parameter along a fourth axis obtained by rotating the second axis by a second angle with respect to the origin.

3. The emotion estimation method according to claim 2,
wherein both the first angle and the second angle are 45 degrees,
the third axis is an axis indicating the excitement degree, and
the fourth axis is an axis indicating the relaxation degree.

4. A content determination method comprising:
obtaining the emotion parameter estimated by the emotion estimation method according to claim 1;
determining content to be provided to the user, based on the emotion parameter obtained; and
outputting information related to the content determined.

5. The content determination method according to claim 4, further comprising:
obtaining a target parameter indicating a target emotion for the user; and
determining the content based on a difference between the emotion parameter and the target parameter.

6. The content determination method according to claim 5, further comprising:
setting, based on the difference, an induction parameter indicating an emotion that the user is induced to feel, to cause the emotion parameter to approach the target parameter; and
determining the content based on the induction parameter.

7. A non-transitory computer-readable recording medium having recorded thereon a computer program for causing a computer to execute the emotion estimation method according to claim 1.

8. A non-transitory computer-readable recording medium having recorded thereon a computer program for causing a computer to execute the content determination method according to claim 4.

9. An emotion estimation system comprising:
an input interface that obtains a first parameter indicating a subjective mood of a user, a second parameter indicating a subjective excitement degree of the user, and a third parameter indicating a subjective relaxation degree of the user;
a signal processing circuit that estimates an emotion parameter indicating an emotion of the user based on the first parameter obtained, the second parameter obtained, and the third parameter obtained; and
an output interface that outputs information related to the emotion parameter.

10. A content determination system comprising:
an input interface that obtains the emotion parameter estimated by the emotion estimation system according to claim 9;
a signal processing circuit that determines content to be provided to the user, based on the emotion parameter obtained; and
an output interface that outputs information related to the content determined.
